(19) United States
(12) Patent Application Publication
(10) Pub. No.: US 2012/0100186 A1
(43) Pub. Date: Apr. 26, 2012

(54) NANOPARTICULATE-BASED CONTRACEPTIVE/ANTI-HIV COMPOSITION AND METHODS

(75) Inventors: Samuel A. WICKLINE, St. Louis, MO (US); Gregory LANZA, St. Louis, MO (US); Joshua HOOD, Valley Park, MO (US)

(73) Assignee: Washington University, St. Louis, MO (US)

(21) Appl. No.: 13/278,049

(22) Filed: Oct. 20, 2011

Related U.S. Application Data
(60) Provisional application No. 61/405,108, filed on Oct. 20, 2010.

(51) Int. Cl. A61K 38/02; A61K 39/395; A61P 31/22; A61P 31/20; A61K 9/00; A61P 31/18; B82Y 5/00

(52) U.S. Cl. 424/400; 514/3.7; 514/3.8; 514/1.1; 424/172.1; 514/4.2; 977/788; 977/915

(57) ABSTRACT

Nanoparticulate compositions which employ membrane-integrating peptides to effect contraception and/or protection against infection by sexually transmitted virus are described.

### CROSS-REFERENCE TO RELATED APPLICATION

This application claims priority from U.S. provisional application 61/405,108 filed 20 Oct. 2010. The contents of this document are incorporated herein by reference.

### FIELD OF THE INVENTION

The invention is in the fields of protection against conception and against HIV infection. More particularly, the invention concerns vaginal preparations that specifically interact with sperm and/or HIV using nanoparticulate delivery systems.

### BACKGROUND ART

There is a well recognized need for protection against HIV transmitted through sexual intercourse as well as an option for contraception, particularly in societies where women have little control over reproduction and sexual interaction. The present invention provides women with means to practice contraception and to protect themselves against HIV infection using a vaginal preparation which can be administered using a simple applicator and does not require cooperation or permission from sexual partners.

The basis for the compositions of the invention resides in perfluorocarbon-based nanoparticles (PFC-NP) that are targeted to sperm or to HIV and that carry a membrane-integrating peptide, i.e., a peptide which forms pores in or lyses cell membranes. U.S. Pat. No. 7,943,168 ('168 patent), incorporated herein by reference, describes such perfluorocarbon nanoparticles which are associated with membrane-integrating peptides. Briefly, the nanoparticles comprise perfluorocarbon cores coated with a lipid/surfactant layer as described, for example, in U.S. Pat. Nos. 7,255,875 and 7,186,399 (the “Lanza patents”), also incorporated herein by reference. The various membrane-integrating peptides that can be associated with the nanoparticles are also described in the above-cited '168 patent and include membrane-lytic peptides and cell-penetrating peptides as well as pore-forming peptides. In particular, melittin and its analogs are described.

As further noted in the above-referenced '168 patent, the nanoparticulates bearing the membrane-integrating peptides may be targeted. Targeting agents can include antibodies, aptamers, peptidomimetics and the like. A description of such targeting agents and means for attachment thereof is also found in the above-referenced Lanza patents as well as U.S. Pat. Nos. 7,255,875, 7,566,442 and 7,344,698, also incorporated herein by reference.

### DISCLOSURE OF THE INVENTION

The invention is directed to compositions designed for application to the vaginal vault which compositions comprise nanoparticles targeted to sperm wherein said nanoparticles further contain membrane-integrating peptides or comprise nanoparticles targeted to sexually transmitted viruses, such as HIV, which nanoparticles further contain membrane-integrating peptides or wherein the composition comprises both. It is desirable, in preventing infection by sexually transmitted viruses for the nanosnares or nanoparticles to be targeted. However, untargeted nanosnares may also be used for this indication. The same nanoparticles may target both sperm and virus.

In another aspect, the invention concerns methods to prevent conception and/or protect a subject against virus infection in a subject which method comprises administering to the vagina of the subject the compositions of the invention.

### BRIEF DESCRIPTION OF THE DRAWINGS

FIGS. 1A, 1B, and 1C show the effect of free melittin as compared to melittin associated with PFC-NP on the viability of vaginal epithelium.

FIG. 2 shows the results of an in vitro experiment whereby HIV infection is prevented by melittin-containing nanoparticles of the invention.

FIGS. 3A and 3B show the effect of melittin coupled PFC-NP on virus infectivity of strains HIV-p120 and HIV-p134.

FIG. 4 is a graph demonstrating the effect of CD4 coupled PFC-NP on coupling of the particles to HIV.

FIGS. 5A-5D show the effect of free melittin or melittin-containing PFC-NP on sperm motility and viability.

FIG. 6 demonstrates that SPAM1 antibody can successfully target sperm.

### MODES OF CARRYING OUT THE INVENTION

In general, “a” or “an” refer to one or more than one of the referent unless the opposite intention is clear from the context.

The compositions of the invention contain thousands of trillions of nanoparticles per intervaginal dose wherein these nanoparticles comprise one or more membrane-integrating peptides. In some embodiments, these nanoparticles, sometimes called herein “nanosnares”, are targeted specifically to sperm or to sexually transmitted viruses, such as HIV. These nanoparticles are typically perfluorocarbon nanoparticles (PFC-NP) and carry a potent toxin in the form of a membrane-integrating peptide that results in the formation of pores in the sperm or virus when these are fused to the nanoparticles. In the case of virus, specific targeting is not necessary since the nanoparticles are substantially larger than the virus particles. Nevertheless, efficiency may be improved by providing a targeting ligand. In the case of sperm, however, targeting is needed for efficient fusion because the fusion event establishes the proximity necessary for formation of a hemi-fusion stalk (<5 nm) in a process driven passively by the energy stored in the lipid membrane of the PFC-NP. Since cells and sperm are a great deal larger than the nanoparticles, non-targeted nanoparticles even comprising multiple copies of the membrane-integrating peptide may not be sufficient to affect the viability of the cells or motility of the sperm. Since only sperm, and not endothelial cells are targeted, nontargeted cells (but not virus) are spared and the nanoparticles in the composition are destructive only to the targeted sperm. As noted above, both targeted and non-targeted particles that comprise the membrane-integrating peptide are effective against virus infections that are sexually transmitted, such as herpes or papillomavirus, or HIV.

To target sperm, the nanoparticles may be associated with a targeting agent for the αᵥβ₃ integrin, which is a well known docking site on the sperm cap. The targeting agent for this integrin may be an antibody specific for the integrin or an immunospecific portion thereof, an aptamer, or may be a peptidomimetic, such as those described in U.S. Pat. No. 7,566,442, incorporated herein by reference. Alternatively, other known sperm-associated receptors can be targeted. In addition to targeting the sperm per se, progesterone can be added to the composition since it is a chemoattractant for sperm that swim up a hormonal gradient sensed through their cap progesterone receptors. Progesterone mimics could also be included as the targeting agent on the nanoparticles.

Targeting agents for sperm also include antibodies or fragments thereof that are specifically immunoreactive with ligands on the surface of the sperm. (“Antibodies”, of course, include any immunoreactive portion of conventional antibodies, including recombinantly produced single chain antibodies, chimeric antibodies, polyclonal antibodies or monoclonal antibodies, antibody mimics, such as aptamers or peptidomimetics and the like.) A particularly useful antibody which might be used, or a fragment of which might be used, is the SPAM antibody marketed by Sigma-Aldrich that is specific for sperm.

For capture of HIV, the targeting ligands may be those that bind to gp41 and/or gp120 epitopes. Here, too, antibodies or aptamers could be employed. Alternatively or in addition CD4, CCR5 and CXCR4 peptides that imitate the viral membrane fusion process for T cells may be used. However, as noted above, effective defense against viral particles in general, including HIV, herpes and papillomavirus may be effected in the vaginal vault using nanosnares containing membrane-penetrating peptides that do not comprise targeting agents.

The composition may include nanoparticles targeted to sperm or nanoparticles targeted to virus or both types of nanoparticles. It is also possible to include targeting ligands to both virus and sperm on the same nanoparticle, or to employ non-targeted nanoparticles for virus protection.

For the targeted nanoparticles useful in the invention, the number of molecules of targeting ligand per nanosnare will vary depending on its nature. However, typically, the number of targeting ligands per nanoparticle is between 10 and 500, alternatively between 20 and 100 or between 20 and 30.

The targeted nanoparticles further comprise toxic membrane-integrating peptides, which are exemplified by melittin. Melittin forms pores in lipid membranes that are too large to be repaired by standard membrane repair mechanisms and thus result in discharge of DNA from sperm or RNA from HIV, rendering both ineffective. This effect is confined in the vaginal vault to the targeted sperm and/or to virus particles for the reasons set forth above, i.e., fusion to the target is needed to effect pore formation in the case of cells as opposed to viruses. In addition, the nanoparticles are too large (100-500 nm, typically 250 nm) to penetrate the vaginal mucosa and thus their action is confined to the vaginal vault and they remain in place until washed away.

As used herein, the word “peptide” is not intended to impose an upper limit on the number of amino acids contained. Any peptide/protein which is capable of effecting cell penetration can be used in the methods of the invention. The nature of the lipid/surfactant layer can be adjusted to provide a suitable environment for the peptides/proteins used in the invention depending on the specific characteristics thereof. Thus, the nature of the lipids and surfactants used in this layer are selected so as to accommodate cationic peptides, anionic peptides, neutral peptides, hydrophobic peptides, hydrophilic peptides, amphipathic peptides, etc.

Membrane-integrating peptides useful in the invention include lytic peptides such as melittin and the classic pore forming peptides magainin and alamethicin (Ludtke, S. J., et al., Biochemistry (1996) 35:13723-13728; He, K., et al., Biophys. J. (1996) 70:2659-2666). Pore forming peptides can also be derived from membrane active proteins, e.g., granulysin, prion proteins (Ramamoorthy, A., et al., Biochim Biophys Acta (2006) 1758:154-163; Andersson, A., et al., Eur. Biophys. J. (2007) DOI 10.1007/s00249-007-0131-9). Other peptides useful in the invention include naturally occurring membrane active peptides such as the defensins (Hughes, A. L., Cell Mol Life Sci (1999) 56:94-103), and synthetic membrane lytic peptides (Gokel, G W., et al., Bioorganic & Medicinal Chemistry (2004) 12:1291-1304). Included as generally synthetic peptides are the D-amino acid analogs of the conventional L forms, especially peptides that have all of the L-amino acids replaced by the D-enantiomers. Peptidomimetics that display cell penetrating properties may be used as well. Thus “cell penetrating peptides” include both natural and synthetic peptides and peptidomimetics.

One particular class of membrane-integrating peptides useful in the invention has the general characteristics of melittin in that it comprises a hydrophobic region of 10-20 amino acids adjacent to a cationic region of 3-6 amino acids. Melittin itself is formed from a longer precursor in bee venom and has the amino acid sequence

(SEQ ID NO: 1)
GlyIleGlyAlaValLeuLysValLeuThrThrFlyLeuPro-
AlaLeuIleSerTrpIleLysArgLysArgGlnGln-NH₂.

Various analogs of melittin can be identified and tested as described in U.S. Pat. No. 5,645,996, for example. Other designs for peptides useful in the invention will be familiar to those in the art. In the melittin analogs, the hydrophobic region is preferably 15-20 amino acids long, more preferably 19-21 and the cationic sequence is preferably 3-5 or 4 amino acids long.

The toxicity of such peptides is affected by a number of factors, including the charge status, bending modulus, compressibility, and other biophysical properties of the membranes as well as environmental factors such as temperature and pH. The presence or absence of certain moieties (other than the targeted epitope) on the cell surface may also effect toxicity.

Illustrated below is the membrane-integrating peptide melittin, which is a water-soluble, cationic, amphipathic 26 amino acid alpha-helical peptide. Suchanek, G., et al., PNAS (1978) 75:701-704. It constitutes 40% of the dry weight of the venom of the honey bee Apis mellifera. Although a candidate for cancer chemotherapy in the past, melittin has proved impractical because of its non-specific cellular lytic activity and the rapid degradation of the peptide in blood. Attempts have been made to stabilize melittin by using D-amino acid constituents (Papo, N., et al., Cancer Res. (2006) 66:5371-5378) and melittin has been demonstrated to enhance nuclear access of non-viral gene delivery vectors (Ogris, M., et al., J. Biol. Chem. (2001) 276:47550-47555 and Boeckle, S., et al., J. Control Release (2006) 112:240-248). The ultimate effect of melittin is to cause the formation of pores in a cell membrane, and membranes of internal cell organelles, so as to damage the cell and lead to cell death. As noted in the examples below, these proteins are also toxic to viruses.

In another embodiment a peptide from the Bcl-2-family proteins is employed based on activating or inhibitory activity, for example, BH3 domain peptides (Danial, N. N., et al., Cell (2004) 116:205-219). After penetrating to the cellular interior the peptides cause activation or inhibition of the endogenous Bcl-2-family or associated proteins in the cells (Walensky, L. D., et al., Mol Cell (2006) 24:199-210). Thus, the cellular machinery of apoptosis can be regulated to a variety of therapeutic goals.

In PFC-NP, the core is inert and nontoxic but facilitates fusion by mobilizing component lipids and relaxing lipid membrane structures.

A variety of means can be employed to couple the targeting agent and the membrane-integrating peptide to the nanoparticles but one advantageous method is through fusion with a peptide linker which is a truncated form of melittin that retains its membrane-binding potential but deletes its lytic capacity. This linking peptide is described in an article by Pan, H., et al., FASEB J. (2010) published online 24 Mar. 2010. This peptide and effective analogs are also described in WO2009/151788, incorporated herein by reference for the description of these peptides and methods for employing these peptides as linkers to couple any desired moiety to the PFC-NP. This linker can be inserted into the lipid layer of the PFC-NP using a 10-minute mixing procedure that drives the peptide to form a hydrophobic interaction with the lipid layer. Alternatively, a component of the lipid/surfactant layer may be used.

However, melittin may simply be passively loaded onto the PFC-NP. The hydrophobic portions of melittin are sufficiently compatible with the lipid/surfactant layer to effect coupling.

Targeted PFC-NP are prepared as described in the above-referenced patents. Targeting ligands to virus or other sperm cell marker that are peptides may be fused to the linker peptide described above to obtain up to 2,000-30,000 total copies of each associated with each nanoparticle. Thus, each of the nanoparticles may also contain about 10-1,000 targeting ligands. Gentle centrifugation removes any unbound ligands. Targeting ligands may be attached to a phospholipid anchor. This is coupled to a component of the lipid/surfactant layer and formulated into the particle itself.

Similarly, a multiplicity of toxin molecules may be associated with the nanoparticles. In the case of melittin, the hydrophobic α-helical portion of the protein serves as a linker whereby the lytic portion is associated with the nanoparticle. Alternative lytic or pore-forming membrane-integrating peptides may be fused to this linker and associated with the nanoparticles as well. The level of toxic pore-forming molecules associated with the nanoparticles can also be varied from just a few to more than 20,000. The pore-forming peptide or lytic peptide may be coupled to a component of the lipid/surfactant layer, as well, in order to associate the toxin with the nanoparticles.

The preparation of successfully derivatized nanoparticles can be verified by means known in the art. For example, flow cytometry may be used to identify and count nanoparticles successfully as associated with targeting ligands and toxins.

Efficacy as a contraceptive may be evaluated in vitro by demonstrating disrupted motility of sperm at selected concentrations of targeted nanoparticles by computer assisted semen analysis and viability of sperm may be tested by dye exclusion and apoptosis staining. Efficacy against virus, such as HIV, may be evaluated by calculating the viral load remaining in the supernatant of a mixture of virus and nanoparticles following 5-30 minute incubations with continuous mixing at 37° C. and low-speed centrifugation to pellet nanoparticle-virus complexes with visual confirmation of complexes by TEM. In addition, targeted nanoparticles incubated in viral cultures are assessed for efficacy of antiviral activity by incubating these cultures with cells that are candidates for viral infection, and observing infection rates.

The nanoparticles described above are formulated into suitable preparations for vaginal administration.

Vaginal Formulations

The nanosnares of the invention are specifically formulated in a composition suitable for vaginal administration. These formulations differ markedly from pharmaceutical compositions in general. Specifically, they are designed to provide a suitable residence time in the vagina and are adjusted for pH and release characteristics that are suitable for this environment. The formulations, when marketed, would be labeled appropriately to limit their use to vaginal administration.

Suitable vaginal preparations may be in the form of aerosols, foams, gels, creams, suppositories or tablets; typically these are in the forms of foams or gels or dissolvable waffles. The excipients in such compositions are typically polyethylene glycols, emulsifying agents, lanolin, starch, algins, polysorbates, xanthan gums, glycerol and the like. Preparation of vaginal compositions is well known in the art and is described, for example, in U.S. Pat. Nos. 5,725,870 and 6,706,276 incorporated herein by reference. Deodorants, colorants and other cosmetic materials may be added as well.

In addition to direct application to the vaginal vault, the vaginal formulations containing the nanosnares of the invention may be applied to condoms. Formulations designed to be retained at the surface of the condom until use are within the skill of the art. Typically, gels or creams can be used for this purpose. This embodiment is especially useful for nanosnares targeted to sperm, an analogy to contraceptive creams that are often applied to condom surfaces. However, the nanospheres designed to inhibit infectivity of sexually transmitted virus may be included as well. The surface may be either the inner or outer surface of the condom or both.

The formulations may contain a single type of nanosnare—i.e., nanosnares that comprise at least one membrane-integrating peptide and which either further comprise a targeting ligand for a sexually transmitted virus, or further comprise a targeting ligand for sperm or do not comprise a targeting ligand or that further comprise both a targeting ligand for sexually transmitted virus and a targeting ligand for sperm or combinations of the foregoing.

Usage

For use, the vaginal preparations of the invention are used in effective amounts. As prepared as a suppository or tablet, typically the suppository or tablet is in the range of 0.1-10 grams or 1-5 grams; as a cream or gel, similar quantities may be employed. The mode of application is dependent on the nature of the composition; for liquid or gel compositions, an applicator is generally employed. Use of coated condoms is also contemplated. The application should be carried out prior to the beginning of vaginal intercourse, generally 1 to 30 minutes, up to 12 hours prior to intercourse. Intermediate times such as 2 hours, 6 hours, etc., are also acceptable. The nature of carriers and excipients and their mode of application is understood in the art.

The following examples are intended to illustrate but not to limit the invention.

Preparation A

Preparation of Perfluorocarbon Nanoparticles

A. Perfluorocarbon nanoparticles were synthesized as described by Winter, P. M., et al., Arterioscler. Thromb. Vasc. Biol. (2006) 26:2103-2109. Briefly, a lipid surfactant co-mixture of egg lecithin (98 mol %) and dipalmitoyl-phosphatidylethanolamine (DPPE) 2 mol % (Avanti Polar Lipids, Piscataway, N.J.) was dissolved in chloroform, evaporated under reduced pressure, dried in a 50° C. vacuum oven and dispersed into water by sonication. The suspension was combined with either perfluoro-octylbromide (PFOB), or perfluoro-15-crown ether (CE) (Gateway Specialty Chemicals, St. Peters, Mo.), and distilled deionized water and continuously processed at 20,000 lbf/in² for 4 min with an S110 Microfluidics emulsifier (Microfluidics, Newton, Mass.) to obtain an emulsion of perfluorocarbon nanoparticles (PFC-NP).

B. Alternatively, a lipid film containing 92.8 mol % lecithin (phosphatidyl choline), 5 mol % cholesterol, and 2.2 mol % MPB-PEG-DSPE was prepared using rotary evaporation. This lipid film representing the 2% surfactant portion was emulsified with sonication in the presence of 20% perfluorocarbon (perfluoro-octyl-bromide, PFOB), 1.85% glycerin and 76.15% water. The emulsion was then prepared into nanoparticles using microfluidization at 20,000 psi. Finished 2 mol % MPB-PEG-DSPE PFOB nanoparticles were sized (281 nm) using dynamic light scattering.

Preparation B

Coupling PFC-NP to Targeting Ligand

αᵥβ₃-integrin targeted nanoparticles were made by incorporating 0.1 mole % peptidomimetic vitronectin antagonist conjugated to polyethylene glycol (PEG)₂₀₀₀-phosphatidylethanolamine (Avanti Polar Lipids, Inc.) replacing equimolar quantities of lecithin in the procedure of Preparation A.

The αᵥβ₃-integrin targeting ligand linked to phosphatidyl ethanolamine has the formula:

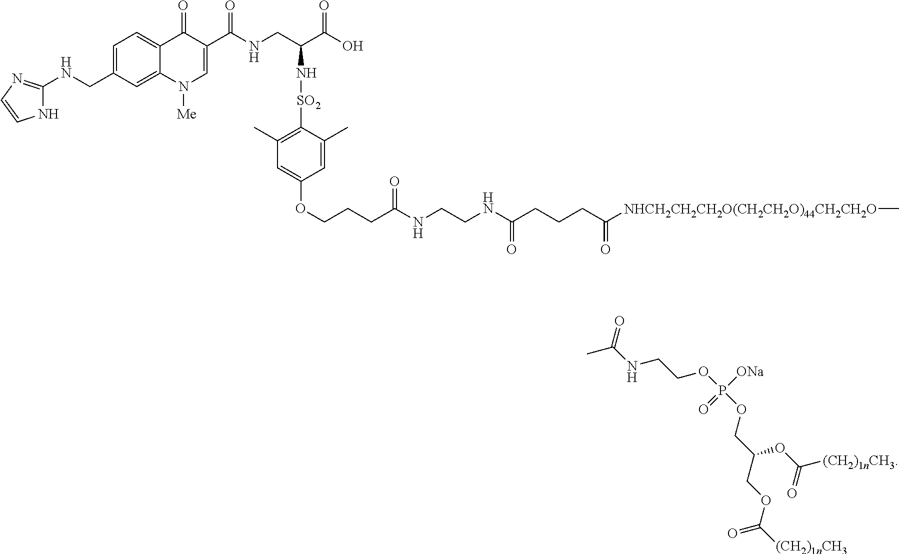

Preparation C

Preparation of Melittin-Containing Nanoparticles

Perfluorocarbon nanoparticles were incubated in a 900 μM solution of melittin at 4° C. protected from light for 3 days. The nanoparticles were then centrifuged at 1000 rpm for 5 minutes and washed with PBS three times. Nanoparticles were stored under argon at 4° C. until use. For comparison, blank nanoparticles of the same concentration were obtained by incubating with PBS rather than melittin according to this protocol.

In more detail, 100 μL of 10 mM melittin or 100 μL of PBS was added to 1000 μL of blank nanoparticles and incubated at 4° C. protected from light with gentle shaking for 3 days.

The level of suspension in the vial used in the mixture is marked and then centrifuged at 1000 rpm for 5 minutes and washed 4× with PBS. The nanoparticles are resuspended to original volume in PBS and stored under argon at 4° C. until use.

In the alternative, melittin was dissolved in 100 mM KCl (pH 7, 10 mM HEPES) at 0.1 mM and 2-20 mL was added to 50 μl of nanoparticle suspension with mixing. After incubation at room temperature for 10 min, the nanoparticles were washed twice by centrifugation (100 g, 10 min) to remove the unbound melittin. The melittin in the supernatant was quantified by measuring the tryptophan fluorescence (described below). Depending on the amount of melittin added, the melittin-loaded nanoparticles yielded molar lipid/melittin ratios ranging from 1,000 to 40.

In still another alternative, the PFC-NP prepared in Preparation A, paragraph B or similar targeted nanosnares were incubated at a concentration of 0.91 mM melittin in water with rotation at 4° C. for 72 hours to load melittin. Nanosnares were isolated by low speed centrifugation for 20 min. at 1000 g to “softly” pellet the nanosnares. Nanosnare supernatants were analyzed for unbound melittin using an Eclipse™ plate reader at excitation 280 nm and emission 300-500 nm Single maximum emission peak sizes corresponding to the amount of melittin present were then compared to a standard 0.91 mM melittin emission peak and used to calculate supernatant and corresponding nanoparticle pellet concentrations of melittin for the nanosnares.

### Example 1

### Vaginal Epithelium Toxicity

In order to function satisfactorily, the compositions of the invention must not be toxic to the vaginal epithelium. This example demonstrates that although free melittin not coupled to nanoparticles is toxic, melittin coupled to untargeted nanoparticles is not toxic at concentrations useful in the invention.

Immortalized vaginal epithelial cells (VK2/E6E7) were obtained from ATCC (CRL-2616) and propagated by the suggested protocol. For toxicity studies, 7500 cells were added to each well of a 96-well plate and allowed to attach for 24 hours. Melittin or melittin coated PFC-NP were then added and incubated with the cells for 12 hours at 37° C. with shaking at 500 rpm. Cells were washed once with media and incubated with MTT reagent for 4 hours. The colored product was solubilized in DMSO and absorbance at 570 nm was measured using a plate reader.

In more detail, cells were detached from a T75 flask by rinsing with 2-3 mL of trypsin, followed by adding 5 mL trypsin and incubating at 37° C. for 15 minutes. Five mL DMEM-FBS is then added to stop the trypsin, and the mixture is centrifuged at 900 rpm for 5 minutes, supernatant is removed and the cells are resuspended in 5 mL of Keratinocyte-Serum Free Media (Invitrogen™). The cells are diluted to approximately 75,000 cells/mL, and 100 μL of the cell suspension is added to each well of a 96-well plate and incubated for 24 hours at 37° C. The media are replaced with 100 μL of media containing the melittin or melittin-loaded nanoparticles at various concentrations and incubated for 12 hours at 37° C. with shaking at 500 rpm. The test solutions are then removed and the cells washed once with media.

To each well containing 100 μL of media, 20 μL of MTT reagent (5 mg/mL MTT in PBS) were added, and incubated for 4 hours at 37° C. The media are removed and 75 μL of DMSO is added to each well and incubated for 10 minutes at 37° C. with shaking at 500 rpm. Absorbance is read at 570 nm using a plate reader.

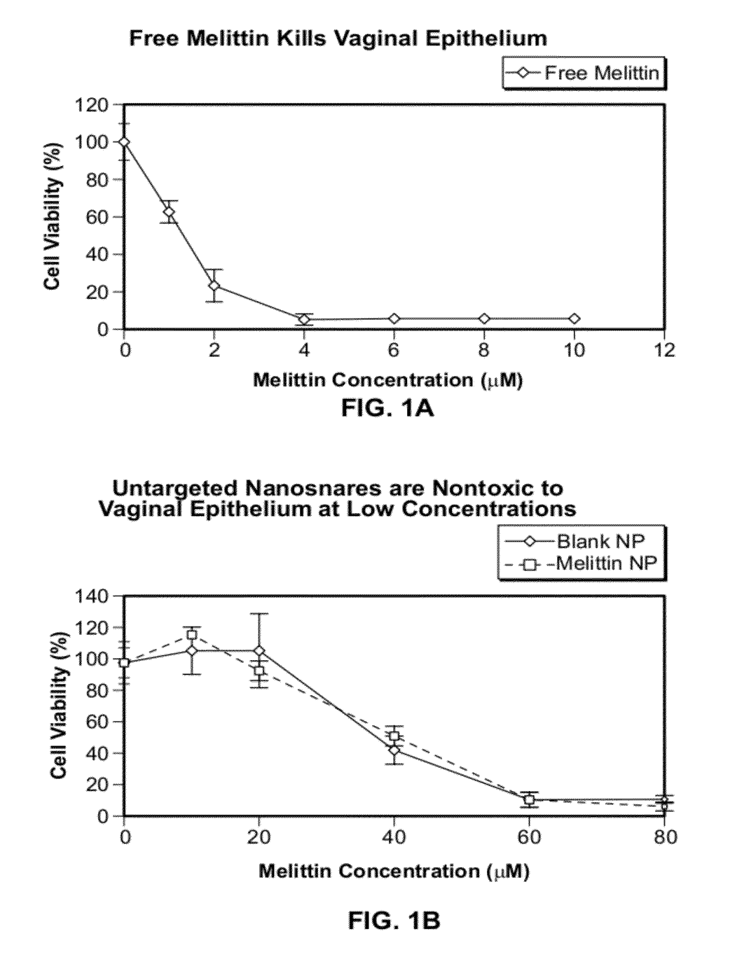

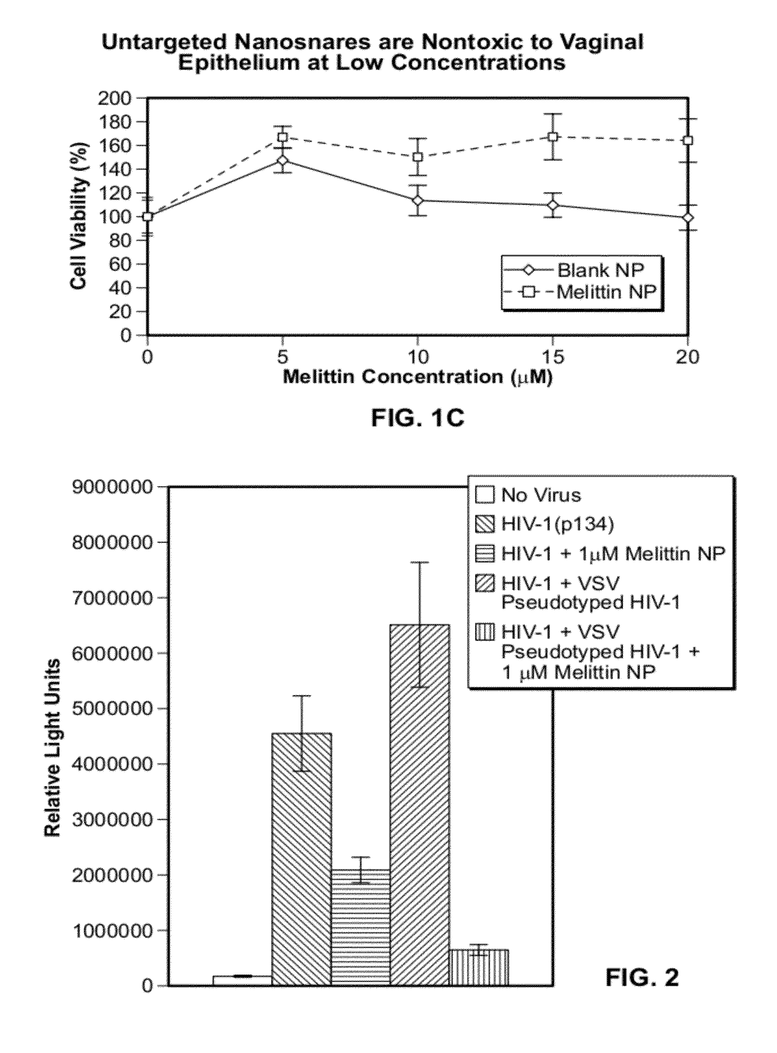

Results are shown in FIGS. 1A-1C. As shown in FIG. 1A, concentrations of melittin as low as 1 μM dramatically decrease the viability of vaginal epithelium cells. However, as shown in FIG. 1B, concentrations of melittin up to 20 μM do not decrease epithelial cell viability. In fact, as shown in FIG. 1C, nanoparticles containing melittin at concentrations up to 20 μM appear to have a somewhat positive effect on cell viability. Controls with nanoparticles not containing melittin (blank NP), but comparable in concentration to the melittin-containing nanoparticles show substantially no effect.

### Example 2

### Contraception Cream

PFC-NP targeted to αᵥβ₃ are prepared as described in U.S. Pat. No. 7,566,442 incorporated herein by reference. Melittin was associated with the particles by dissolving it at a concentration of 0.1 mM in 100 mM KCl, pH 7 and 0-20 ml is added to 50 μl of the nanoparticle suspension. After incubation at room temperature for 10 minutes, the nanoparticles are washed twice by centrifugation to remove unbound melittin. The melittin-loaded nanoparticles yield molar lipid/melittin ratios ranging from 1,000-40.

One gram of the particles thus prepared is added to a mixture of polyethylene glycol 400, polyethylene glycol 6,000 and hexantriol to form a water-soluble vaginal cream.

### Example 3

### Fusogenic Anti-HIV “Nanosnares” Decrease Infectivity of HIV to TZM-b1 Cells

Vesicular stomatitis virus (VSV) pseudotyped HIV-1 is a strain of HIV that demonstrates several fold increases in the levels of transfection over amphotropic HIV by utilizing an endocytic entry mechanism. TZM-b1 cells are specially engineered HeLa cells designed to express CD4 and either CXCR4 or CCR5 which are required for HIV fusion. The cells also contain HIV-TAT inducible luciferase. When TZM-b1 cells are infected, firefly luciferase is produced. Cells to be assessed for infection are lysed and luciferin is added. Fluorescence is produced by infected cells, and the level of fluorescence, in relative fluorescence units, corresponds to the level of viral infection.

In this assay, PFC-NP (25 μl) providing 1 μM melittin in a nanoparticle/virus solution and virus (HIV or VSV pseudotyped HIV) (50 μl) were mixed, and then added to 4×10⁴ cultured cells in 50 well plates and incubated overnight at 37° C. The level of infection was measured as described above.

The results are shown in FIG. 2. As shown, when HIV alone or VSV pseudotyped HIV alone was added to the cells, high levels of infection occurred. However, the level of viral infectivity of either virus combined with the melittin-containing nanoparticles of the invention is dramatically mitigated.

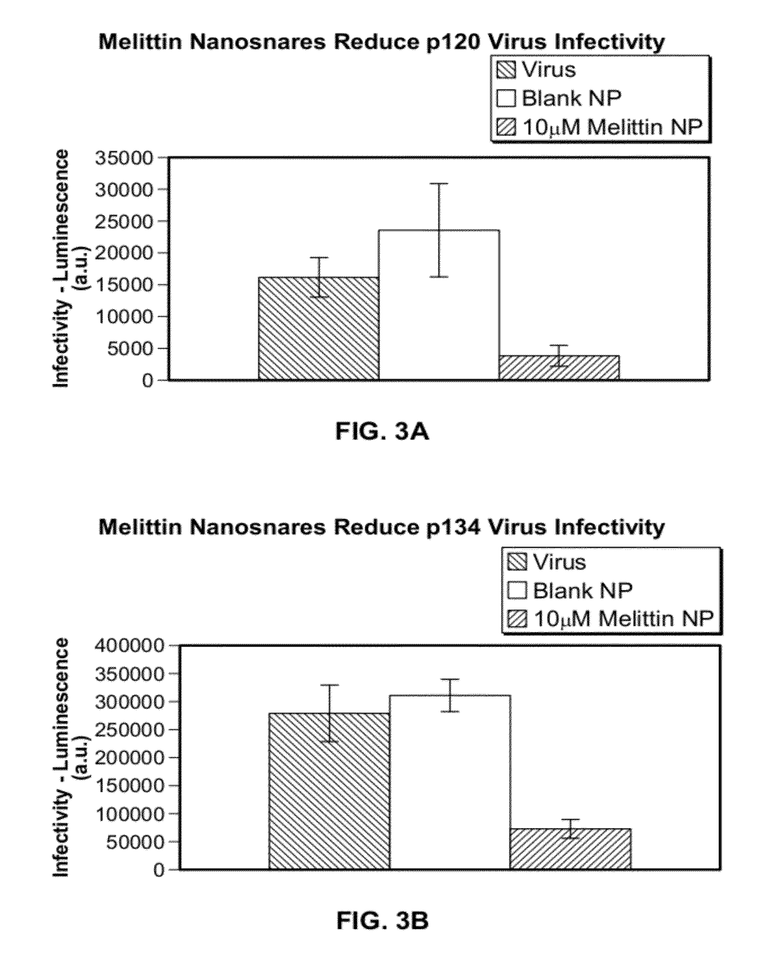

The procedure set forth above was repeated comparing HIV-1p120 which is a CXCR4-dependent strain and p134 which is a CCR5 dependent strain. These results are shown in FIG. 3A and FIG. 3B. As shown, the infectivity of either p120 or p134 in the presence of nanoparticles alone (blank nanoparticles) is not effectively reduced; however, in the presence of melittin-containing nanoparticles with an effective concentration of 10 μM melittin, infectivity is dramatically decreased.

### Example 4

### Effect of Targeted Nanosnares

The nanosnares of this example were prepared as follows.

A lipid film containing 92.8 mol % lecithin (phosphatidyl choline), 5 mol % cholesterol, and 2.2 mol % MPB-PEG-DSPE was prepared using rotary evaporation. This lipid film representing the 2% surfactant portion was emulsified with sonication in the presence of 20% perfluorocarbon (perfluoro-octyl-bromide, PFOB), 1.85% glycerin and 76.15% water. The emulsion was then prepared into nanoparticles using microfluidization at 20,000 psi. Finished 2 mol % MPB-PEG-DSPE PFOB nanoparticles were sized (281 nm) using dynamic light scattering.

For preparation of the CD4 anti-HIV targeting motif, 300 μg CD4 (Pro Sci recombinant sCD4)/0.0111 μmoles was dissolved in 2.1 ml of 0.1 M PBS pH 8.0 then mixed with 5 μL of 7.3 nM 2-iminothiolane in 0.1 M PBS, 10 mmol EDTA buffer pH 8.0, flushed with argon gas and incubated at RT for 1 hour. For conjugation of CD4 to the nanoparticles, 15 ml of 2 mol % MPB-PEG-DSPE PFOB was added into 2.1 ml of modified CD4 solution and incubated at room temperature for 2 hours. Then 7 mg cysteine was added to quench MPB reactivity and incubated for another 2 hours. Finally, the nanoparticle suspension was then dialyzed in 2 L PBS buffer three times at intervals of 2, 3, and 2 hours to remove free unreacted sCD4 and excess 2-iminothiolane thus producing CD4 nanosnares.

To produce melittin loaded CD4 nanosnares, CD4 nanosnares were incubated at a concentration of 0.91 mM melittin in water with rotation at 4° C. for 72 hours to load melittin. Nanosnares were isolated by low speed centrifugation for 20 min. at 1000 g to “softly” pellet the nanosnares. Nanosnare supernatants were analyzed for unbound melittin using an Eclipse™ plate reader at excitation 280 nm and emission 300-500 nm. Single maximum emission peak sizes corresponding to the amount of melittin present were then compared to a standard 0.91 mM melittin emission peak and used to calculate supernatant and corresponding nanoparticle pellet concentrations of melittin for the nanosnares. Melittin only nanosnares were created using the same method in the absence of sCD4. “Blank” nanosnares were created using the same method without sCD4 or melittin.

In the resulting products PFC-NP comprising melittin contained about 20,000 melittin molecules per nanosnare and PFC-NP coupled to CD4 contained about 20-30 CD4 molecules per nanosnare.

HIV-1 producing 293T cell supernatants were harvested 48 h postlipofection, filtered, and assayed for p24 antigen content by enzyme-linked immunosorbent assay. Viruses were resuspended in culture media, aliquoted and stored at −80° C. Equal amounts of virus based on p24 content were used in each experiment.

HIV-1p134 viral strain was coincubated with blank nanospheres, melittin-bearing nanospheres, CD4-labeled nanospheres or CD4-melittin containing nanospheres at the same concentration levels of nanoparticles in each case. Ninety (90) μL of virus were added to 10 μL of the various nanoparticulate stocks. The final concentration with regard to nanosnares containing melittin is 0.06 mM melittin. To test whether the virus particles can be captured by the nanospheres, advantage is taken of the comparatively large size of the nanoparticles as compared to virus such that the nanoparticles are segregated by centrifugation at 1000×G. To detect capture of the virus, the level of the viral protein p24 in the supernatant and precipitate was compared. The initial p24 value was the same for every sample, confirming that the same level of virus particles was present in each sample.

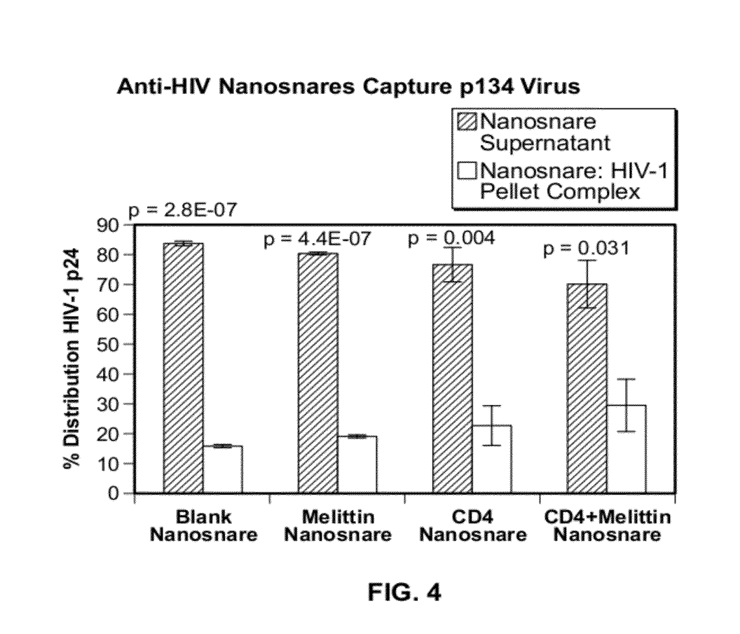

As shown in FIG. 4, CD4-containing nanospheres show a larger percentage of p24 in the precipitate as compared to supernatant in contrast to nanospheres that do not contain CD4.

### Example 5

### Effect of Melittin-Containing Particles on Sperm Motility and Viability

Human semen samples were obtained from the Washington University in vitro fertilization laboratory and stored at 37° C. until use. Semen was diluted in EmbryoMax® Human Tubal Fluid (HTF) (Millipore) and free melittin or melittin-coupled nanoparticles in HTF was added to achieve a final sperm concentration of 10 million sperm per mL. (For nanoparticles, samples were pipetted up and down 3 times every 10 minutes to keep the nanoparticles suspended.) Samples were incubated in 96-well plates for 30 minutes at 37° C. with shaking at 150 rpm to prevent sperm from settling. Afterwards, Viadent® stain (Hoechst 33258, Hamilton Thorne) was added to achieve a final stain concentration of 10 μg/mL and samples were incubated for an additional 5 minutes. Sperm motility and viability were determined using an IVOS® Computer Assisted Sperm Analyzer (Hamilton Thorne).

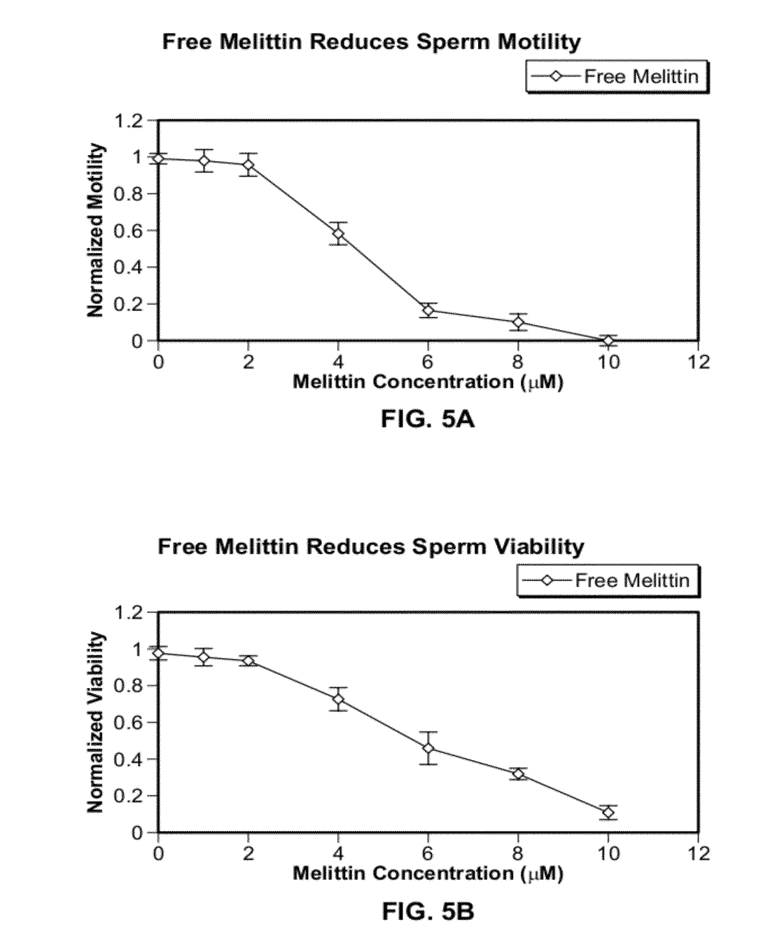

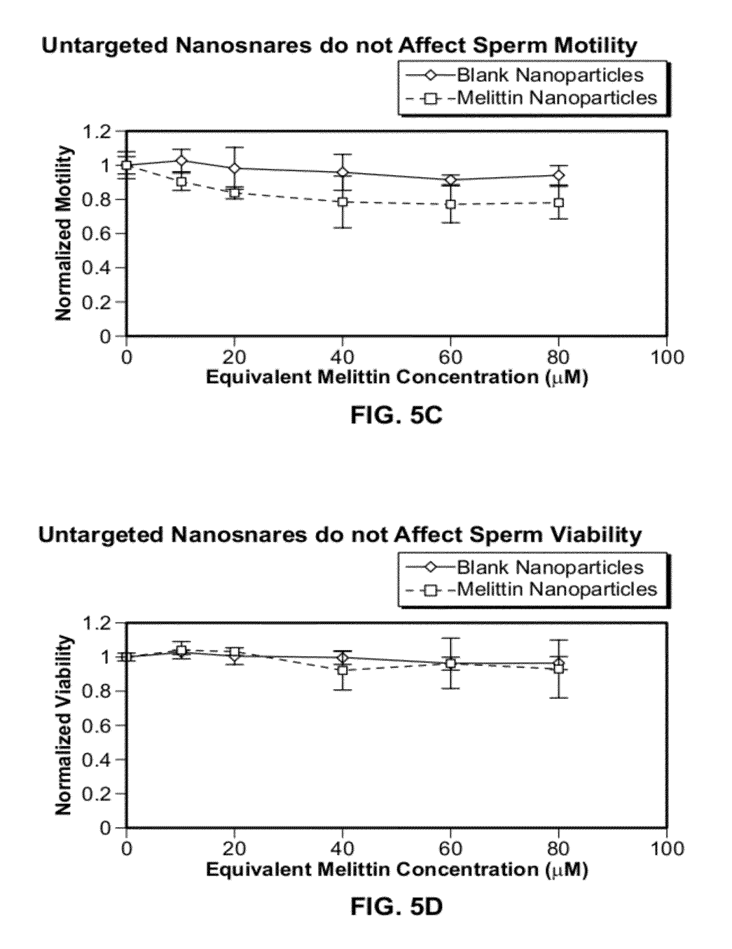

The results are shown in FIGS. 5A-5D. As shown, both sperm viability and sperm motility are impeded in free melittin, but the presence of melittin on nanoparticles in comparable amounts shows no effect. The ability of nanoparticles to protect sperm (and endothelial cells) but not viruses from melittin poisoning is explained by the difference in size of the virus as compared to sperm or epithelial cells. These results indicate that sperm targeting is needed to effect inhibition of motility and viability of sperm. Means to provide sperm-targeting are illustrated in the next example.

### Example 6

### Successful Sperm Targeting

This example shows successful sperm targeting by the sperm-specific antibody SPAM1 (Sigma) as compared to control rabbit IgG (Thermo Scientific).

Sperm samples were centrifuged at 400 g for 5 minutes, the supernatant removed and the cells were fixed with 4% paraformaldehyde for 20 minutes at room temperature.

Sperm were again centrifuged at 400 g for 5 minutes, the supernatant removed and the pellet blocked in PBS, 2% BSA, 5% normal serum (serum of the animal in which secondary antibody was generated) for 60 minutes at room temperature. 0.5% Triton® X-100 was added if needed to permeabilize the sperm.

Sperm were again centrifuged at 400 g. The supernatant was removed and incubated overnight at 4° C., suspended in PBS, 2% BSA containing either the anti-SPAM1 antibody (Sigma-Aldrich, 1:100 dilution, 2 μg/mL) or control rabbit IgG (Thermo Scientific, 2 μg/mL).

After washing the pellet three times in PBS, 2% BSA at room temperature, the pellets were incubated in PBS, 2% BSA containing the appropriate secondary antibody (e.g., Alexa Fluor®-488 goat anti-rabbit IgG, Invitrogen, 1:250 dilution) or TO-PRO® dye (Invitrogen, 1:500 dilution, 2 μM) for 45 minutes at 4° C., and again washed three times in PBS at room temperature.

Five μL of the stained solutions were placed on a slide and smeared by passing another glass slide over the surface, and covered with a cover slip containing 15 μL of Vectashield® (Vector Labs), sealed with nail polish and visualized using confocal microscopy (60× objective).

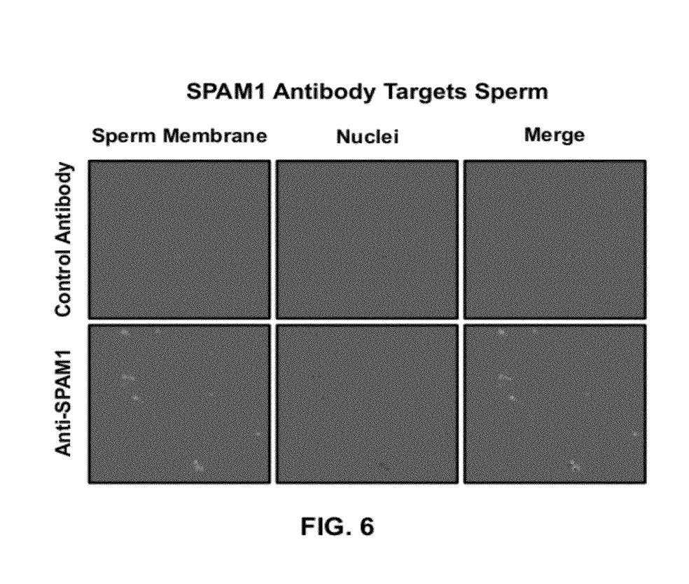

The results are shown in FIG. 6 demonstrating that the SPAM antibody successfully targets sperm.

1. A composition formulated for vaginal administration, which composition comprises perfluorocarbon-based nanoparticles (PFC-NP) wherein said PFC-NP comprise at least one membrane-integrating peptide,
and wherein said nanoparticles:
a) further comprise at least a targeting ligand for a sexually transmitted virus,
b) further comprise at least a targeting ligand for sperm, or
c) do not comprise a targeting ligand.

2. The composition of claim 1 which is in the form of a gel, foam, cream, suppository, or a dissolvable waffle.

3. The composition of claim 1 which is coated on a condom.

4. The composition of claim 1 wherein the nanoparticles further comprise a targeting ligand for sperm and wherein if the membrane-integrating peptide is melittin, said targeting ligand is other than a ligand targeting αᵥβ₃.

5. The composition of claim 4 wherein the targeting ligand comprises an antisperm antibody or fragment thereof or is an aptamer.

6. The composition of claim 1 wherein said nanoparticles further comprise a targeting ligand for a sexually transmitted virus.

7. The composition of claim 6 wherein the targeting ligand targets HIV and comprises a targeting ligand for gp120, gp41, or a targeting ligand which is CD4, CXCR4 or CCR5.

8. The composition of claim 7 wherein the targeting ligand is CD4.

9. The composition of claim 1 wherein the membrane-integrating peptide is a pore-forming peptide.

10. The composition of claim 9 wherein the pore-forming peptide is melittin or an analog thereof.

11. The composition of claim 4 which further contains progesterone.

12. A method to prevent conception and/or viral infection in a subject which method comprises administering vaginally to said subject an effective amount of the composition of claim 1.

13. A method to prevent conception in a subject which method comprises administering vaginally to said subject an effective amount of the composition of claim 4.

14. A method to prevent viral infection in a subject which method comprises administering vaginally to said subject an effective amount of the composition of claim 6.

15. A method to prevent sexually transmitted viral infection in a subject which method comprises administering vaginally to said subject an effective amount of a composition that comprises PFC-NP comprising a membrane-integrating peptide.

16. The method of claim 15 wherein the viral infection is infection by HIV, herpes or papillomavirus.
